(19) United States
(12) Patent Application Publication
(10) Pub. No.: US 2015/0093447 A1
(43) Pub. Date: Apr. 2, 2015

(54) STEM CELL IDENTIFICATION AND PURIFICATION METHOD

(71) Applicant: NEOSTEM, INC., NEW YORK, NY (US)

(72) Inventors: David O'Neil, New York, NY (US); Yajuan Jiang, Mahwah, NJ (US); Elizabeth Leary, Haverhill, MA (US)

(21) Appl. No.: 14/387,467

(22) PCT Filed: Mar. 25, 2013
(86) PCT No.: PCT/US13/33766

§ 371 Date: Sep. 23, 2014

Related U.S. Application Data
(60) Provisional application No. 61/615,154, filed on Mar. 23, 2012.

(51) Int. Cl. A61K 35/48; A61K 35/14; C12N 5/074; G01N 33/569

(52) U.S. Cl. CPC A61K 35/51; G01N 33/56966; A61K 35/14; C12N 5/0607
USPC 424/577; 435/7.21; 435/6.1

(57) ABSTRACT

The invention provides a composition comprising enriched extracellular vesicles, wherein the extracellular vesicles express VSEL markers. The extracellular vesicles are used to treat or regenerate damages or injured tissue in a subject.

### CROSS-REFERENCE TO RELATED APPLICATIONS

This application claims priority to U.S. Application No. 61/615,154, filed Mar. 23, 2012, which is incorporated herein by reference in its entirety.

### FIELD OF THE INVENTION

The present invention relates to isolation and purification of VSEL stem cell populations, extracellular vesicles and similar enucleated particles expressing markers of VSELs, and to the use of such stem cells and microparticles for treatment.

### BACKGROUND OF THE INVENTION

Peripheral blood and mobilized peripheral blood comprise many different types of cells and other components, including stem cells such as, but not limited to, very small embryonic-like stem cells (VSELs), mesenchymal stem cells (MSCs) and hematopoietic stem cells (HSCs). VSELs are a population of small (4 to 8 μm) cells that express CD133, CD34 and/or CXCR4, as well as markers characteristic of embryonic stem cells such as Oct-4 and Nanog. Molecular and in vitro and in vivo functional studies indicate that VSELs are pluripotent and capable of regenerating damaged host tissues. Such subpopulations of Lin⁻CD45⁻ cells have been identified in human umbilical cord blood as well as in adult human bone marrow and G-CSF mobilized peripheral blood (PB).

### SUMMARY OF THE INVENTION

The present invention provides methods and compositions for the separation or fractionation of stem cells and particles displaying stem cell markers. To isolate human VSELs for further characterization from G-CSF mobilized adult PB, a 4-color panel of antibodies (anti-CD45 Pacific Blue, CD34-PE, CD133-PE, FITC-conjugated antibodies to WBC, RBC and PLT lineage markers) and the cell viability dye 7-AAD was used to track the cells during purification. Events with the VSEL phenotype (live Lin⁻CD45⁻CD34/133⁺ flow cytometry events) are relatively rare, approximately 1 for every 40 CD45^{mid}CD34⁺ hematopoietic progenitor cells. However, using methods that separate cells based on size or density such as differential centrifugation, percoll gradient centrifugation, and counterflow centrifugal elutriation, it was observed that the Lin⁻CD45⁻CD34/133⁺ events fall into two separate populations with different physical characteristics—a major population (approximately 98% of Lin⁻CD45⁻CD34/133⁺ events) of objects that are very small (<4 μm), very light, and that stain negatively or dimly with the nuclear dye DRAQ5, and a minor population that is larger (5-10 μm), heavier, and that stains brightly with DRAQ5. FACS sorting of the two populations followed by cytospin and diff-quick stain showed that the minor population consists of small nucleated cells, whereas the major population consists of membrane-bound objects that do not have a cell nucleus. By light microscopy and transmission electron microscopy these objects have the appearance of extracellular vesicles. Although most are roughly the size of platelets, their morphologic appearance is quite different from platelets. The two populations of Lin⁻CD45⁻CD34/133⁺ events are also found in umbilical cord blood, although at different frequencies than in mobilized adult blood (1 for every 5 hematopoietic progenitors, with 94% of the events being DRAQ5⁻). Accordingly, a nuclear marker such as DRAQ5 is useful to quantify and distinguish cells having the VSEL phenotype by flow cytometry. As with the DRAQ5+ nucleated VSELs, the enucleated particles which express markers of VSELs can be purified and used as therapy to repair damaged or injured tissues in human subjects.

The invention provides a composition comprising enriched extracellular vesicles, wherein the extracellular vesicles comprise CD45⁻/lin⁻/CD34⁺, or CD45⁻/lin⁻/CD133⁺, or CD45⁻/lin⁻/CD34⁺/CD133⁺. The extracellular vesicles are enucleated and are substantially unstained by a membrane permeant nuclear dye, such as DRAQ5, thiazole orange or LDS-751. In an embodiment of the invention, the extracellular vesicle composition is substantially free of nucleated cells.

In certain embodiments, the extracellular vesicles comprise CXCR4+. In certain embodiments, the extracellular vesicles further comprise at least one of SSEA-4, Oct-4, Rev-1, and Nanog. In certain embodiments, the extracellular vesicles further comprise a lineage-related marker.

The extracellular vesicles of the invention encompass a range of sizes smaller and are generally smaller than VSELs. In embodiments of the invention, the extracellular vesicles have a diameter of 40-100 nm, 50-150 nm, 100-300 nm, 200-500 nm, 400-1000 nm, 500-1500 nm, 1-2 μm, 2-3 μm, 3-4 μm, or 4-5 μm

The invention also provides a pharmaceutical composition comprising the enriched extracellular vesicles.

The invention also provides a method for treating damage or injury to a tissue in a subject, comprising administering to the subject an effective amount of a pharmaceutical composition comprising extracellular vesicles of the invention.

The invention further provides a method of making a composition enriched for extracellular vesicles which express VSEL markers, which comprises a) providing a population comprising CD45⁻ extracellular vesicles, b) identifying a subpopulation of CD45⁻ extracellular vesicles that expresses a marker of VSEL stem cells, and c) purifying the subpopulation. According to the invention, the method produces extracellular vesicles which comprise CD45⁻/lin⁻/CD34⁺, or CD45⁻/lin⁻/CD133⁺, or CD45⁻/lin⁻/CD34⁺/CD133⁺. In certain embodiments, the extracellular vesicles comprise CXCR4⁺. In certain embodiments, the extracellular vesicles comprise at Least one of SSEA-4, Oct-4, Rev-1, and Nanog. In certain embodiments, the extracellular vesicles comprise a lineage-related marker. In certain embodiments, the composition is substantially free of nucleated cells.

The invention also provides method of purifying a very small embryonic-like (VSEL) stem cell, which comprises a) providing a population comprising CD45⁻ VSEL stem cells, b) identifying a subpopulation of CD45⁻ stem cells that expresses a marker of VSEL stem cells and stains with a vital nuclear dye, and c) purifying the subpopulation. The method provides VSEL compositions of increased purity which are substantially free of enucleated VSEL-like components, such as extracellular vesicles. In certain embodiments, the vital nuclear dye is DRAQ5, thiazole orange or LDS-751. In certain embodiments, the purification method involves identifying and purifying the CD45⁻ subpopulation which comprises CD45⁻/lin⁻/CD34⁺, or CD45⁻/lin⁻/CD133⁺, or CD45⁻/lin⁻/CD34⁺/CD133⁺. In certain embodiments, the purification method involves identifying and purifying the CD45⁻ subpopulation which comprises CXCR4⁺. In certain embodiments, the purification method involves identifying and purifying the subpopulation which comprises at least one of SSEA-4, Oct-4, Rev-1, and Nanog. In certain embodiments, the purification method involves identifying and purifying the subpopulation which comprises a lineage-related marker.

### DESCRIPTION OF THE FIGURES

FIG. 1 shows useful 4- and 5-color cocktails, the 5-color cocktail comprising DRAQ5 that can be used for flow cytometry analysis of cell fractions according to the invention.

FIG. 2 shows that a subpopulation of cells identified by VSEL markers is also stained by the nucleic acid dye DRAQ5. In the 5-color assay depicted, no nucleated (DRAQ5) Lin⁻CD45⁻CD34/133⁺ cells are detectable in apheresis start material or elutriation fractions without further enrichment.

FIG. 3 shows DRAQ5⁺ labeled cells are detectable in cell fractions enriched for CD34/133⁺ from Elutra fractions 70 or higher (here fractions 70 and 110).

FIG. 4 shows subpopulations of cells isolated as VSELs can be distinguished by DRAQ5 staining A) VSEL 4-color assay. B) VSEL 5-color (DRAQ5) assay. The 5-color results typically have low event counts (typically <1 VSEL per million events in all fractions) C) Apheresis VSELs showing proportion of VSELs stained by DRAQ5. DRAQ5⁻ VSELs are less dense (don't pellet at 400× gravity), than DRAQ5⁺ VSELs (can pellet at 400 or even 100×g). DRAQ5⁻ VSELs are about 50× more prevalent than DRAQ5⁺ VSELs in G-CSF-mobilized peripheral blood.

FIG. 5 shows VSEL content in various cell fractions. The starting materials were: Elutra 1, 400×g supernatant; Elutra 2, 100×g supernatant; Elutra 3, 100×g pellet. Most VSELs detected by 4-color assay are light and small. VSELs identified by 5-color assay are bigger (elutriate later) and heavier (pellet more easily by centrifugation).

FIG. 6 depicts cell processing methods used herein. RBCs were removed by Ficoll or lysis. The processing methods include elutriation directly from an apheresis product and elutriation of pelleted, ficoll-purified cells. Also depicted is an immunomagnetic selection strategy using the EasySep system from StemCell Technologies.

FIG. 7 shows 5-color VSEL events at the limits of detection in apheresis start material and standard elutriation fractions. DRAQ5⁻ VSELs (4-color) are more common that the DRAQ5⁺ VSELs, and elutriate earlier. The y-axis indicates numbers of VSEL events per million counted flow cytometry events.

FIG. 8 shows destination of red blood cells (RBC), white blood cells (WBC) and platelets (PLT) following pelleting and RBC lysis. The PLT stay in the 400 g sup, WBC and RBC go the 400 g pellet. RBC disappear after lysis.

FIG. 9 shows enrichment from Elutra 2 fraction 70 (from 100×g supernatant) by immunomagnetic selection. RBCs have been lysed. CD34⁺ cells (top center of each plot) are enriched by three different methods, with EasySep CD34⁺ giving the best enrichment.

FIG. 10 shows the lin⁻ gate for the samples depicted in FIG. 9.

FIGS. 11-14 show enrichment of DRAQ5⁻ VSEL-like vesicles (non-nucleated vesicles with VSEL surface staining characteristics). The arrows show the enriched populations of vesicles in the CD34-selected fraction. FIG. 11: 400×g supernatant, Elutra fraction 35; FIG. 12: 400×g supernatant; FIG. 13: 400×g supernatant) by immunomagnetic selection. There was an 800-fold enrichment of VSELs away from the major platelet contaminant in this fraction. FIG. 14: same as FIG. 13, showing the 5-color (DRAQ5⁺) nucleated VSEL population. Most of the VSEL-like events purified from 400×g supernatant are DRAQ5⁻.

FIG. 15 shows the proportion of DRAQ5⁺ nucleated VSEL events for CD34 enriched objects from the 400×g supernatant (<1% DRAQ5+), Elutra fraction 35 (<1% DRAQ5+), and Elutra fraction 70 (>75% DRAQ5+).

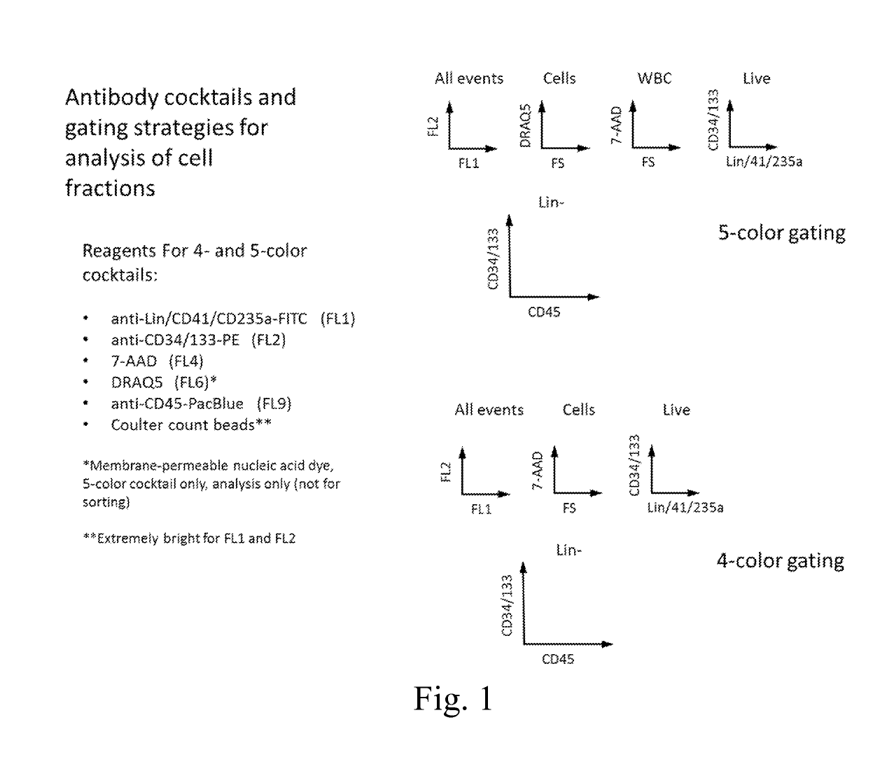

### DETAILED DESCRIPTION

In one embodiment, the present invention relates to a method for isolating one or more populations of stem cells in at least one step from a peripheral blood or mobilized peripheral blood sample. In particular, in one embodiment, the present invention relates to a method for the flow-rate separation of stem cell populations using elutriation (sized-based separation) to separate the peripheral blood into various fractions based on cell size, each enriched for a different stem cell or vesicle of interest. These populations are characterized, for example, by cell surface markers, and nucleic acid content. Different fractions or subfractions comprise an increase in proportion of Very Small Embryonic Like Stem cells (VSELs), MSCs or HSCs as compared to a non-fractionated apheresis product.

The invention provides purified VSEL stem cells or a cell-like particle characterized by Lin⁻/CD45⁻/CD34/133⁺, or Lin⁻/CD45⁻/CD34/⁺ of Lin⁻/CD45⁻/CD34/133⁺ (VSEL markers), and impermeable to 7-amino-actinomycin D (7-AAD), which has a high DNA binding constant and is efficiently excluded by intact cells. Murine VSEL stem cells and cell-like particles comprise Lin⁻/CD45⁻/Sca-1⁺. The VSELs are comparatively larger (5-10 μm), heavier (pellets at 400×g), nucleated (DRAQ5 bright, nucleus visible by light microscopy), and relatively rare (about 80,000 per mobilized apheresis). The particles are comparatively small (1-3 μm), relatively light (<1.07 g/ml, does not pellet at 400×g), show no clear evidence of a nucleus by flow cytometry (DRAQ5^{lo/-}) or by microscopy, and are more abundant (about 3 million per mobilized apheresis). The smaller, apparently enucleated particles are referred to herein as “extracellular vesicles.”

The extracellular vesicles may contain nucleic acids, including but not limited to mRNA, tRNA, microRNA, and the like, but can be understood to be substantially unstained by a dye that preferentially labels DNA. Substantially unstained means that the extracellular vesicles are stained to a sufficiently low level that they are distinguishable from nucleated cells, such as VSELs. Also, with respect to an extracellular vesicle, the term “express” or “expresses” means the extracellular vesicle contains or presents a protein, nucleic acid, or surface marker, even though the extracellular vesicle might not possess the capacity to make such components.

The VSEL stem cells and extracellular vesicles can be obtained and isolated or purified by a variety of procedures, for example on the basis of their size, density, and staining characteristics (e.g., surface markers, lack of staining by nuclear dyes). In one embodiment, the VSELs and/or extracellular vesicles are obtained from peripheral blood by apheresis. In an embodiment of the invention, the VSELs and/or extracellular vesicles are obtained from mobilized peripheral blood. In another embodiment, the VSELs and/or extracellular vesicles are obtained from cord blood. In another embodiment, the VSELs and/or extracellular vesicles are obtained from bone marrow. In another embodiment, the VSELs and/or extracellular vesicles are obtained from spleen. In another embodiment, the VSELs and/or extracellular vesicles are obtained from adult tissue. In another embodiment, the VSELs and/or extracellular vesicles are obtained from adipose tissue.

As provided herein, separation and purification procedures can be employed that distinguish cells and other cell-like objects that comprise VSEL stem cell markers. More particularly, the invention provides an enrichment method or method step, by which rare VSEL stem cells are distinguished from more abundant enucleated particles of similar size, which also bear markers characteristic of VSELs. Further, the invention identifies and provides a method of isolating and purifying enucleated VSEL-like extracellular vesicles from separation fractions and preparations containing VSELs, as well as from separation fractions and preparations not previously known to include such particles.

Thus, whereas VSELs can be obtained by apheresis and elutriation, and are enriched in certain of the resulting purification fractions, more abundant (DRAQ5⁻) VSEL-like objects can now be purified from such fractions, as well as from fractions not previously identified as containing VSEL-like particles. For example, VSEL-like microparticles can be obtained and enriched from 400×g apheresis supernatant and elutriation fractions equivalent to Elutra fraction 35. In certain embodiments, VSEL-like microparticles are enriched by immunomagnetic selection, including, but not limited to EasySep CD34⁺ kit and other selection reagents described herein.

In some embodiments, the isolated VSEL stem cell or VSEL stem cell-like populations obtained by the methods as disclosed herein, can be maintained in appropriate buffers or culture medium and/or cryopreserved for future use, e.g., for use alone, or selectively recombined (e.g., custom mixing) for individualized autologous therapeutic applications in regenerative therapy. In some embodiments, the isolated VSEL stem cells may be expanded prior to use or cryopreservation.

Additionally, in some embodiments, the isolated stem cell populations also provide a pool of different stem cell populations for personalized cell-based assays to assess the effect of a person's diet, pharmacogenetics, neutrochemicals, and lifestyle on the function and viability of different stem cell populations, either alone or as a combination of different stem cells.

In some embodiments, the peripheral blood fractionation is achieved by elutriation, wherein the flow rate through the elutriation apparatus determines which cells from the peripheral blood are collected in a specific PB fraction. In some embodiments, an apheresis product from peripheral blood is processed at different flow rates through an Elutra® apparatus, or other suitable elutriation apparatus or machine. In some embodiments, the flow rate used in the elutriation procedure is maintained and centrfugal acceleration force (i.e., “g”) is varied to fractionate the peripheral or apheresed blood.

According to the invention, a cell fractionation method comprises the use of a membrane-permeable nucleic acid dye to identify and/or quantify cellular components. In certain embodiments, the nucleic acid dye is one that can be used in live or dead cells in combination with other fluorescent markers, including, but not limited to, FITC tags and fluorescent proteins. In certain embodiments, subpopulations of cells, such as stem cells, are identified using the nucleic acid dye in combination with one or more cell surface markers or cellular characteristics. In certain embodiments, cell populations, such as stem cell populations, identified by particular combinations of surface markers and other characteristics are subdivided according to staining by the nucleic acid dye. For example, a stem cell type otherwise identified or purified on the basis of four markers is identified or purified using those markers in combination with a nucleic acid dye. In the examples disclosed herein, the nucleic acid is DRAQ5. Nucleic acid dyes that can be used according to the invention further include, without limitation, thiazole orange and LDS-751. Certain dyes have different emission or absorbance spectra when bound to DNA vs. RNA, and can be used to distinguish RNA- or DNA-containing objects.

DRAQ5 is a far-red fluorescent membrane-permeable nuclear stain that can be used in live and or fixed cells in combination with other common fluorophores. Other non-limiting examples of membrane-permeable nuclear stains that can be used in the invention are thiazole orange and LDS-751. According to the invention, such stains may be used together with one or more membrane-impermeable dyes to quantify viable nucleated cells. In one embodiment, DRAQ5 is used with 7-AAD (which is excluded by viable cells but can penetrate cell membranes of dying or dead cells). In another embodiment, DRAQ5 is used with forward scatter to quantify stem cells.

In certain embodiments, a subpopulation that stains well with the nucleic acid dye is collected. In other embodiments, a subpopulation of cells and/or cell-like particles that are stained poorly or remain essentially unstained by the nucleic acid dye are identified or isolated. In certain embodiments, subpopulations of cells and/or cell-like particles are isolated on the basis of the combined use of nucleic acid stains and other markers. For example, a particular subpopulation once identified in a particular elutriation fraction using the combination of markers can continue to be isolated from the same elutriation fraction without further use of some or all of the markers.

Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including method for isolating and culturing them, may be found in, among other places, Embryonic Stem Cells, Methods and Protocols, Turksen, ed., Humana Press, 2002; Weisman et al., Annu Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735. Descriptions of stromal cells, including methods for isolating them, may be found in, among other places, Prockop, Science, 276:71 74, 1997; Theise et al., Hepatology, 31:235 40, 2000; Current Protocols in Cell Biology, Bonifacino et al., eds., John Wiley & Sons, 2000 (including updates through March, 2002); and U.S. Pat. No. 4,963,489.

The term “mesenchymal stem cell” is also referred to herein as “MSC” and refers to multipotent stem cells capable of differentiating into more than one specific type of mesenchymal or connective tissue (i.e., tissues of the body which support specialized elements; e.g., adipose, osseous, stroma, cartilaginous, elastic and fibrous connective tissues). Human mesenchymal stem cells (hMSCs) are reactive with certain monoclonal antibodies, known as SH2, SH3 and SH4. (See U.S. Pat. No. 5,486,359, which is incorporated herein in its entirety by reference). MSCs can be differentiated from HSCs based on their immunospecific profiles and, with MSCs being SH2+/CD14− and human HSCs SH2−/CD14+. For purposes of identification, human MSCs can be identified based on (i) phenotypic marker expression of CD34−, CD45−, CD90+, CD105+ and CD44+, (ii) functional phenotype, including the ability to form colony forming units in a CFA assay as disclosed in the Examples herein, and ability to differentiate into tissues which support specialized elements, including but not limited to: chondrocytes, cartilage and adipocytes. Other markers expressed by MSCs are known in the art and include without limitation CD71, CD73, Stro-1, and CD166, and CD271. In certain embodiments, MSCs are lin⁻.

The term “very small embryonic-like stem cell” is also referred to herein as “VSEL stem cell” and refers to pluripotent stem cells. In some embodiments, the VSEL stem cells (“VSELs”) are human VSELs and may be characterized as lin⁻, CD45⁻, and CD34⁺. In some embodiments, the VSELs are human VSELs and may be characterized as lin⁻, CD45⁻, and CD133⁺. In some embodiments, the VSELs are human VSELs and may be characterized as lin⁻, CD45⁻, and CXCR4⁺. In some embodiments, the VSELs are human VSELs and may be characterized as lin⁻, CD45⁻, CXCR4⁺, CD133⁺, and CD34⁺. In some embodiments, human VSELs express at least one of SSEA-4, Oct-4, Rex-1, and Nanog, and possess large nuclei surrounded by a narrow rim of cytoplasm, and contain embryonic-type unorganized chromatin. With respect to stem cell markers, mouse VSELs express at least one of SSEA-1, Oct-4, Rex-1, and Nanog. VSELs also have high telomerase activity. In some embodiments, the VSELs are human VSELs and may be characterized as lin⁻, CD45⁻, CXCR4⁺, CD133⁺, Oct 4⁺, SSEA4⁺, and CD34⁺. In some embodiments, the human VSELs may be less primitive and may be characterized as lin⁻, CD45⁻, CXCR4⁺, CD133⁻, and CD34⁺. In some embodiments, the human VSELs may be enriched for pluripotent embryonic transcription factors, e.g., Oct-4, Sox2, and Nanog. Apart from their deficiency in nuclear material, in embodiments of the invention, the VSEL stem cell-like extracellular vesicles of the invention may be characterized by the same surface markers (including surface markers as well as protein and mRNA content). In some embodiments, the human VSELs may have a diameter of 4-5 μm, 4-6 μm, 4-7 μm, 5-6 μm, 5-8 μm, 6-9 μm, or 7-10 μm. In some embodiments, the VSEL stem cell-like extracellular vesicles man have a diameter of 40-100 nm, 50-150 nm, 100-300 nm, 200-500 nm, 400-1000 nm, 500-1500 nm, 1-2 μm, 2-3 μm, 3-4 μm, or 4-5 μm.

These extracellular vesicles can be further fractionated to obtain subpopulations that express one or more lineage-related markers, including, but not limited to neural cells, skeletal muscle cells, cardiac cells, liver cells, intestinal epithelium cells, pancreas cells, endothelium cells, epidermis cells, and melanocytes. For example, VSEL extracellular vesicles can be fractionated using reagents that detect neural tissue-related glial fibrillary acidic protein (GFAP), nestin, β III tubulin, oligodendrocyte transcription factor 1 (Olig1), and/or oligodendrocyte transcription factor 2 (Olig2). Similarly, VSEL extracellular vesicles can be fractionated using reagents that detect the expression of skeletal muscle-related Myf5, MyoD, and/or myogenin. Additional VSEL extracellular vesicle types and markers that can be employed include, but are not limited to cardiomyocyte-related VSEL extracellular vesicles (Nsx2.5/Csx, GATA-4), liver cell-related VSEL extracellular vesicles (α-fetoprotein, CK19), intestinal epithelium-related VSEL extracellular vesicles (Nkx 2-3, Tcf4), pancreas cell-related VSEL extracellular vesicles (Nkx 6.1, Pdx 1, C-peptide), endothelial cell-related VSEL extracellular vesicles (VE-cadherin), epidermal cell-related VSEL extracellular vesicles (Krt 2-5, Krt 2-6a, BNC), and melanocyte-related VSEL extracellular vesicles (DCT, TYR, TRP).

In one embodiment of the present invention, the VSEL or extracellular vesicle populations collected from the peripheral blood of a subject by the methods as disclosed herein can be introduced or transplanted back to the individual when the subject is in need of such therapy.

A composition comprising a VSEL or extracellular vesicle population collected from the peripheral blood of a subject by the methods as disclosed herein can be used to repair, treat, or ameliorate various aesthetic or functional conditions (e.g., defects) through the augmentation of damaged tissues. The populations collected from the peripheral blood of a subject by the methods as disclosed herein provide an important resource for rebuilding or augmenting damaged tissues, and thus represent the ability to collect medically useful VSEL or extracellular vesicle from a subject from a single source. In a preferred embodiment, the VSEL or extracellular vesicle populations collected from the peripheral blood of a subject by the methods as disclosed herein can be used in tissue engineering and regenerative medicine for the replacement of body parts that have been damaged by developmental defects, injury, disease, or the wear and tear of aging. The VSEL or extracellular vesicle populations collected from the peripheral blood of a subject by the methods as disclosed herein therefore provide significant advantages for individualized stem cell therapy.

The VSEL or VSEL-like extracellular vesicle populations of the invention have therapeutic utility for cardiac repair, and can have homing capacity. Accordingly, the VSEL or VSEL-like extracellular vesicle populations can be formulated for direct injection into cardiac tissue, or formulated for systemic administration. The VSELs are capable of expansion and differentiation, as well as promoting regeneration by myocardial and endothelial progenitor cells. The VSEL-like extracellular vesicle populations promote migration and differentiation of cardiomyocyte precursors, as well as blood vessel formation by endothelial cells and precursor cells.

In another embodiment, the invention provides a method of treating acute and chronic wounds, including without limitation, burns and abrasions and wounds resulting from skin removal e.g., for grafting, or to remove a cancerous lesion, chronic ischemic skin lesions; scleroderma ulcers; arterial ulcers; diabetic foot ulcers; pressure ulcers; venous ulcers; nonhealing lower extremity wounds; ulcers due to inflammatory conditions, and/or long-standing wounds, by administering a composition comprising a VSEL or extracellular vesicle population provided herein. When administered in an effective amount, the VSEL or extracellular vesicle population enhances healing, for example by increased migration of cells from the wound site. In certain embodiments, the VSEL or extracellular vesicle population can be allogeneic. Such allogeneic compositions can be matched between donor and recipient, and the compositions may optionally comprise an immunosuppressant. Extracellular vesicles are advantageous where migration of host cells is desired, and repopulation of the wound by stem cells is unnecessary or undesirable.

The use of a nuclear stain in VSEL isolation and purification methods provides a means of purifying intact, vital cells. Methods of treatment us pharmaceutical compositions comprising such cells. In certain embodiments, the number of VSELs in such a composition can be from 10 to 10², or from 10² to 10³, or from 10³ to 10⁴, or from 10⁴ to 10⁵, or from 10⁵ to 10⁶, or more VSELs. In other embodiments, the number of VSELs employed can be related to the volume or the surface area to be treated. In an embodiment of the invention, wherein the composition is applied over an area of tissue, the composition can comprise from about 50 to about 50,000 VSELs per cm². In another embodiment, the composition comprises about 200 to about 10,000 VSELs per cm². In an embodiment of the invention, the composition comprises from about 500 to about 5,000 VSELs per cm².

Similarly, in certain embodiments, the number of VSEL extracellular particles can be from 10² to 10³, or from 10³ to 10⁴, or from 10⁴ to 10⁵, or from 10⁵ to 10⁶, or from 10⁶ to 10⁷, or more. In an embodiment of the invention, wherein the composition is applied over an area of tissue, the composition can comprise from about 500 to about 500,000 VSEL extracellular particles per cm². In another embodiment, the composition comprises about 2000 to about 100,000 VSEL extracellular particles per cm². In an embodiment of the invention, the composition comprises from about 5000 to about 50,000 VSEL extracellular particles per cm².

The selected dosage level will depend on the activity of the therapeutic composition, the route of administration, and the severity of the condition being treated. However, it is within the skill of the art to adjust dose to until the desired effect is achieved.

In certain embodiments, VSEL or extracellular vesicle population is used to treat osteochondral tissue, particularly bone or cartilage. The VSEL or extracellular vesicle population can be administered directly to the tissue to be treated. In other embodiments, the VSEL or extracellular vesicle population is incorporated into a matrix, or scaffold, which may be biodegradable. In certain embodiments, the matrix is selected to elicit differentiation of the VSELs towards the desired tissue type. In certain embodiments, the matrix is selected to elicit migration of host cells in response to VSEL extracellular vesicles. In certain embodiments, the VSEL or extracellular vesicle population is administered with an agent or growth factor that promotes differentiation towards the desired tissue type. In certain embodiments, the growth factor is a bone morphogenic protein (BMP). In certain embodiments, the method involves providing VSEL or extracellular vesicle populations in a composition that is used to fill or coat a defect in an osteochondral tissue. The number of VSEL or extracellular vesicle population in the composition that is employed can be related to the volume or the surface area of the defect.

The invention also provides methods and compositions for treating damage or an injury to ocular tissue. According to the invention, a composition comprising an effective amount of a VSEL or extracellular vesicle population is administered to the ocular tissue, in order to regenerate or repair the tissue. In one embodiment, the tissue is retinal tissue. In certain embodiments, the VSELs differentiate at the site of repair. In other embodiments, a VSEL extracellular vesicle population induces differentiation and or repair by cells in the tissue or that migrate to the tissue. In certain embodiments, the VSEL or extracellular vesicle population is administered directly to ocular tissue to be treated, for example, but not limited to, subretinally. In other embodiments, the VSEL or extracellular vesicle population is administered by intravitreal injection. In certain embodiments, the VSEL or extracellular vesicle population is incorporated into a matrix, or scaffold, which may be biodegradable.

In addition, such VSEL or extracellular vesicle populations collected from the peripheral blood of a subject by the methods as disclosed herein and compositions thereof can be used for augmenting soft tissue not associated with injury by adding bulk to a soft tissue area, opening, depression, or void in the absence of disease or trauma, such as for “smoothing.” Multiple and successive administrations of the VSEL or extracellular vesicle populations collected from the peripheral blood of a subject by the methods as disclosed herein are also embraced by the present invention.

In another embodiment, the invention provides a method of treating radiation exposure in a subject, which comprises collecting a VSEL and/or extracellular vesicle population from the subject, and administering an effective amount of VSELs and/or VSEL extracellular vesicles to treat the radiation exposure. In an embodiment of the invention, the subject is administered an agent to mobilize VSELs prior to collection. The collected VSELs may be expanded and/or directed or selected to differentiate prior to administration to the subject, and VSEL extracellular vesicles may be collected during expansion and differentiation steps. In one embodiment, an expanded population of VSEL stem cell-derived cells capable of differentiation into hematopoietic/lymphopoietic stem cells is produced and administered to the subject. In another embodiment, VSEL extracellular vesicles are administered to the subject. In certain embodiments, a radiation exposure victim is treated with autologous VSELs and/or VSEL-derived cells and/or VSEL extracellular vesicles. In certain embodiments, a radiation exposure victim is treated with allogeneic VSELs and/or VSEL-derived cells and/or VSEL extracellular vesicles.

For treatment purposes, VSEL or extracellular vesicle populations collected from the peripheral blood of a subject by the methods as disclosed herein are preferably collected from an autologous or heterologous human or animal source. An autologous animal or human source is more preferred. The compositions are then prepared and isolated as described herein. To introduce or transplant a VSEL or extracellular vesicle population collected from the peripheral blood of a subject by the methods as disclosed herein and/or compositions thereof according to the present invention into a human or animal recipient, a suspension of cells or extracellular vesicles is prepared. Such suspensions contain concentrations of a target population collected from the peripheral blood of a subject by the methods as disclosed herein in a physiologically-acceptable carrier, excipient, or diluent. Alternatively, stem cell or extracellular vesicle suspensions may be in serum-free, sterile solutions, such as cryopreservation solutions. The suspensions may then be introduced, e.g., via injection, into one or more sites of the donor tissue.

Concentrated or enriched VSELs or extracellular vesicles may be administered as a pharmaceutically or physiologically acceptable preparation or composition containing a physiologically acceptable carrier, excipient, or diluent, and administered to the tissues of the recipient organism of interest, including humans and non-human animals. The composition may be prepared by resuspending the cells or extracellular vesicles in a suitable liquid or solution such as sterile physiological saline or other physiologically acceptable injectable aqueous liquids. The amounts of the components to be used in such compositions can be routinely determined by those having skill in the art.

In some embodiments, the VSEL or extracellular vesicle populations collected from the peripheral blood of a subject by the methods as disclosed herein or compositions thereof may be administered by placement of the suspensions onto absorbent or adherent material, e.g., a collagen sponge matrix, and insertion of the material into or onto the site of interest. Alternatively, the VSEL or extracellular vesicle populations collected from the peripheral blood of a subject by the methods as disclosed herein may be administered by parenteral routes of injection, including subcutaneous, intravenous, intramuscular and intrasternal. Other modes of administration include, but are not limited to, intranasal, intrathecal, intracutaneous, percutaneous, enteral, and sublingual. In one embodiment, VSEL or extracellular vesicle population collected from the peripheral blood of a subject by the methods as disclosed can be administered by endoscopic surgery.

For injectable administration, a composition comprising a VSEL or extracellular vesicle population collected from the peripheral blood of a subject by the methods as disclosed herein can be suspended in a sterile solution or suspension or may be resuspended in pharmaceutically- and physiologically-acceptable aqueous or oleaginous vehicles, which may contain preservatives, stabilizers, and material for rendering the solution or suspension isotonic with body fluids (i.e., blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

Consistent with the present invention, a population of VSEL or extracellular vesicles collected from the peripheral blood of a subject by the methods as disclosed herein can be administered to body tissues, including epithelial tissue (e.g., skin, lumen, etc.) muscle tissue (e.g., smooth muscle), blood, brain, and various organ tissues such as those organs that are associated with the urological system (e.g., bladder, urethra, ureter, kidneys, etc.).

According to the general treatment method described herein, a VSEL or extracellular vesicle population collected from the peripheral blood of a subject by the methods as disclosed herein can comprise other cells, (e.g., a fraction of cells comprising a target stem cell population) and can be administered to a subject, for example, by infusion into the blood stream of a subject through an intravenous (i.v.) catheter, like any other i.v. fluid. Alternatively, however, an individualized mixture of cells may be generated so as to provide a cellular therapy mixture specific for therapeutic needs of a subject. The comprehensive mixture of cells obtained such as through an apheresis process may be characterized, sorted, and segregated into distinct cell populations. Cell markers such as VSEL markers or tissue specific markers may be used to phenotypically characterize the populations of cells collected from the peripheral blood. Using these markers, it is possible to segregate and sort on the basis of cell type. The mixture of cells or extracellular vesicles is thus transformed into populations of cells, which may be broadly classified into two portions: a stem cell or extracellular vesicle portion and a non-stem cell portion. The non-stem cell portion may further be classified into a progenitor cell or fibroblast portion and a functional cell or fully differentiated cell portion. Once the peripheral blood cellular mixture is sorted, the stem cell or extracellular vesicle portion and non-stem cell portions may be cryopreserved and stored separately. In this manner, a library or repository of distinct cell populations from a subject may be created. Alternatively, stem cell or extracellular vesicle portion and non-stem cell portion may the cryopreserved together and then sorted and separated prior to use.

In some embodiments, the VSEL or extracellular vesicle population collected from the peripheral blood of a subject by the methods as disclosed herein can be used to generate or differentiate a population of a cell type that developed from a germ layer (i.e., endoderm, mesoderm, and ectoderm). These include, but are not limited to, differentiated cells, neural progenitor or differentiated cells, glial progenitor or differentiated cells, oligodendrocyte progenitor or differentiated cells, skin progenitor or differentiated cells, hepatic progenitor or differentiated cells, muscle progenitor or differentiated cells, bone progenitor or differentiated cells, mesenchymal stem or progenitor cells, pancreatic progenitor or differentiated cells, progenitor or differentiated chondrocytes, stromal progenitor or differentiated cells, cultured expanded stem or progenitor cells, cultured differentiated stem or progenitor cells, or combinations thereof. Of interest are hematopoietic cells, which may include any of the nucleated cells which may be involved with the erythroid, lymphoid or myelomonocytic lineages, as well as myoblasts and fibroblasts. Also of interest are progenitor cells, such as hematopoietic, neural, stromal, muscle (including smooth muscle), hepatic, pulmonary, gastrointestinal, and mesenchymal progenitor cells. Also of interest are differentiated cells, such as, osteoblasts, hepatocytes, granulocytes, chondrocytes, myocytes, adipocytes, neuronal cells, pancreatic, or combinations and mixtures thereof.

In some embodiments, a VSEL or extracellular vesicle population collected from the peripheral blood of a subject by the methods as disclosed herein can be combined, recombined, or compounded into a cellular therapy mixture of cells appropriate for treating the disease of a subject and/or regenerating a specific tissue. A combination of VSEL or extracellular vesicle population collected from the peripheral blood of a subject by the methods as disclosed herein, tissue specific progenitor cells, and optionally functional cells can be used, for example, to enhance the engraftment of the transplanted cells. Accordingly, in one embodiment, the present invention provides methods and products for using an autologous mixture of one or more VSEL or extracellular vesicle populations collected from the peripheral blood of a subject by the methods as disclosed herein, either alone, or in combination with other functional cells to enhance engraftment of the transplanted stem cells. In some embodiments, a cellular therapy product may comprise: from about 10% to about 90% of a specific VSEL or extracellular vesicle population collected from the peripheral blood of a subject by the methods as disclosed herein, about 10% to about 80%, or about 10% to about 60%, or about 10% to about 40%, or about 10% to about 90%, of a VSEL or extracellular vesicle population collected from the peripheral blood of a subject by the methods as disclosed herein. In some embodiments, a VSEL or extracellular vesicle population can also comprise a non-stem cell population, such as about from about 5% to about 50% functional cells, about 5% to about 40% functional cells, about 5% to about 30% functional cells, about 5% to about 20% functional cells, or about 5% to about 10% functional cells. Accordingly, in some embodiments, the isolated VSEL or extracellular vesicle populations provide a library of different stem cell types from a particular individual which can be maintained in culture and/or cryopreserved for future use, e.g., for use alone, or selectively recombined (e.g., custom mixing) for individualized autologous therapeutic applications in regenerative therapy.

In another aspect of the present invention, a VSEL or extracellular vesicle populations collected from the peripheral blood of a subject by the methods as disclosed herein can be stored in a cell bank to support an elective healthcare insurance model to effectively protect members of the population from future diseases. An individual subject can elect to have his or her own VSEL or extracellular vesicle populations collected from the peripheral blood, processed and preserved, while he or she is in healthy state, for future distribution for his or her healthcare needs.

Accordingly, in one embodiment, VSEL or extracellular vesicle populations collected from the peripheral blood of a subject by the methods as disclosed herein are “banked” for future use, at a stem cell bank or depository or storage facility, or any place where the target stem cell populations are kept for safekeeping. The storage facility may be designed in such a way that the populations collected from the peripheral blood of a subject by the methods as disclosed herein are kept safe in the event of a catastrophic event such as a nuclear attack. In some embodiments, the storage facility might be underground, in caves or in silos. In other embodiments, it may be on the side of a mountain or in outer space. The storage facility may be encased in a shielding material such as lead.

In the use of a VSEL or extracellular vesicle population obtained by the methods as disclosed herein, the population is contacted with the agent of interest, and the effect of the agent assessed by monitoring output parameters, such as expression of markers, cell viability, differentiation characteristics, multipotent capacity and the like. The VSEL or extracellular vesicle population may be freshly isolated using the methods as disclosed herein, or in some embodiments, cultured, cryopreserved, or genetically engineered prior to using in an assay. The VSEL or extracellular vesicle population can be environmentally induced variants of clonal cultures: e.g., split into independent cultures and grown under distinct conditions, for example, with or without virus; in the presence or absence of other cytokines or combinations thereof. Alternatively, the VSEL or extracellular vesicle population may be variants with a desired pathological characteristic. For example, as the target stem cells are from the subject (i.e., autologous) they have a desired pathological characteristic, e.g., mutation and/or polymorphism which contribute to disease pathology.

Optionally, the VSEL or extracellular vesicle population can be manipulated to express desired gene products. Gene therapy can be used to either modify a cell to replace a gene product or add or knockdown a gene product. In some embodiments, the genetic engineering is done to facilitate regeneration of tissue, to treat disease, or to improve survival of the cells or extracellular vesicles following implantation into a subject (e.g., prevent rejection).

A skilled artisan could envision a multitude of genes which would convey beneficial properties to the transfected mesenchymal cells or, more indirectly, to the recipient stem cells and/or subject if the stem cells are used in transplantation (discussed in more detail below). The added gene may ultimately remain in the recipient cell and all its progeny, or may only remain transiently, depending on the embodiment. For example, genes encoding angiogenic factors could be transfected into progenitor cells isolated from smooth muscle. Such genes would be useful for inducing collateral blood vessel formation as the smooth muscle tissue is regenerated. It some situations, it may be desirable to transfect the cell with more than one gene.

In some instances, it is desirable to have the gene product secreted. In such cases, the gene product preferably contains a secretory signal sequence that facilitates secretion of the protein. For example, if the desired gene product is an angiogenic protein, a skilled artisan could either select an angiogenic protein with a native signal sequence, e.g., VEGF, or can modify the gene product to contain such a sequence using routine genetic manipulation (see, e.g., Nabel et al., 1993).

The desired gene can be transfected into the cell using a variety of techniques. Preferably, the gene is transfected into the cell using an expression vector. Suitable expression vectors include plasmid vectors (such as those available from Stratagene, Madison Wis.), viral vectors (such as replication defective retroviral vectors, herpes virus, adenovirus, adenovirus associated virus, and lentivirus), and non-viral vectors (such as liposomes or receptor ligands).

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

It must be noted that as used herein and in the appended claims, the singular forms “a,” “and,” and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

### Sorting Strategies Using Membrane-Permeable Nucleic Acid Dyes

Membrane-permeable nucleic acid dyes can be used together with membrane-impermeable dye to quantify viable nucleated cells. Examples include: Guava ViaCount assay (LDS-751 vs PI); Nexcelom Cellometer (AO vs PI); DRAQ5 vs 7-AAD.

Membrane-permeable nucleic acid dyes can be viewed vs. forward scatter (FS) to quantify RBC, PLT, and WBC. Examples include thiazole orange vs. FS; and DRAQ5 vs FS.

A typical blood sample was separated using an Elutra Cell Separation System. Elutra fraction 35 contained large amounts of PLTs, fraction 70 mostly RBC and WBC, and fractions greater than 70 mostly WBC, followed by RBC.

### Example 2

### DRAQ5⁺ Staining of VSELs

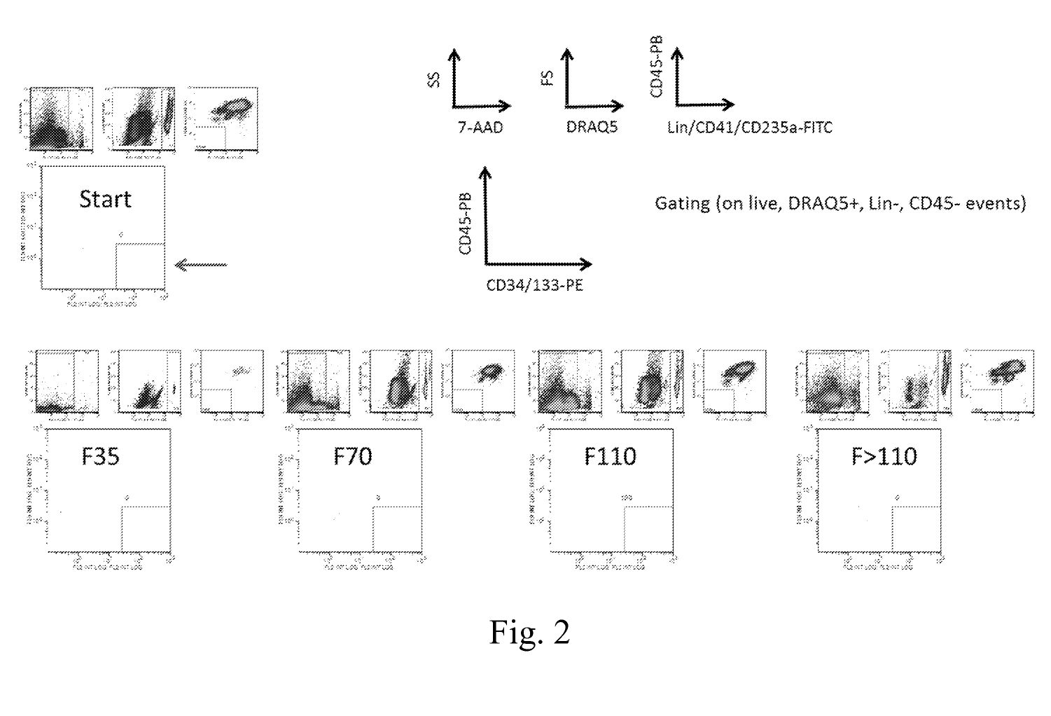

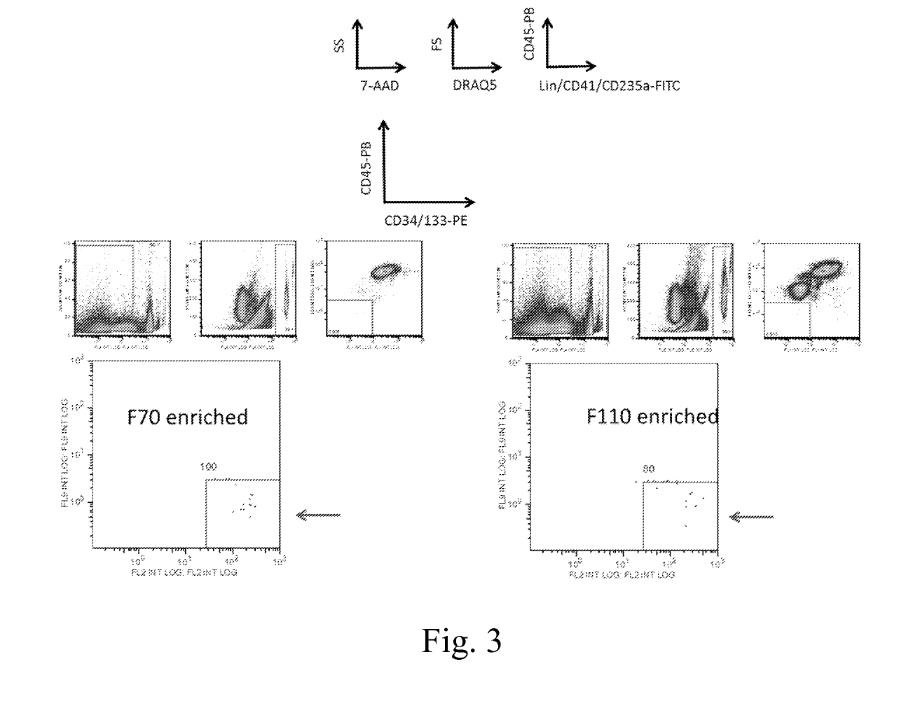

A test subject was mobilized with G-CSG, cells were obtained by apheresis, separated by elutriation, and examiner for VSEL markers. FIG. 2 shows that no nucleated (DRAQ5) Lin⁻CD45⁻CD34/133⁺ cells are detectable in apheresis start material or elutriation fractions without further enrichment. After MACS enrichment for CD34/CD133⁺, nucleated (DRAQ5⁺) Lin⁻CD45⁻CD34/133⁺ cells are detectable in later elutriation fractions (FIG. 3).

It was observed that the vast majority of VSEL-like events (i.e., objects identified as having VSEL markers) do not stain well with DRAQ5. Notably, VSEL-like events in Elutra fractions 20/35 stained poorly with DRAQ5. The density of these VSEL-like particles (<1.07 g/ml) is similar to the density of PLTs.

The vast majority of VSEL events elutriated with PLTs in fractions 20/35. Very pure, viable nucleated (DRAQ5⁺) VSELs were evident in MACS/FACS-sorted elutriation fractions 50-70, although in very low numbers (3,000-30,000 per apheresis product; 1 per million events or less).

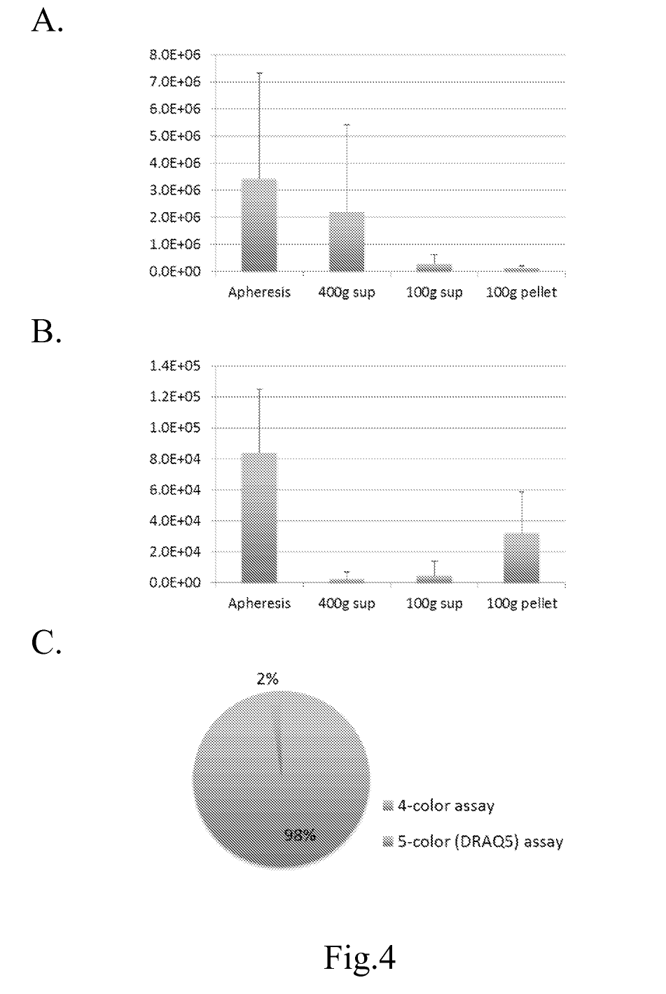

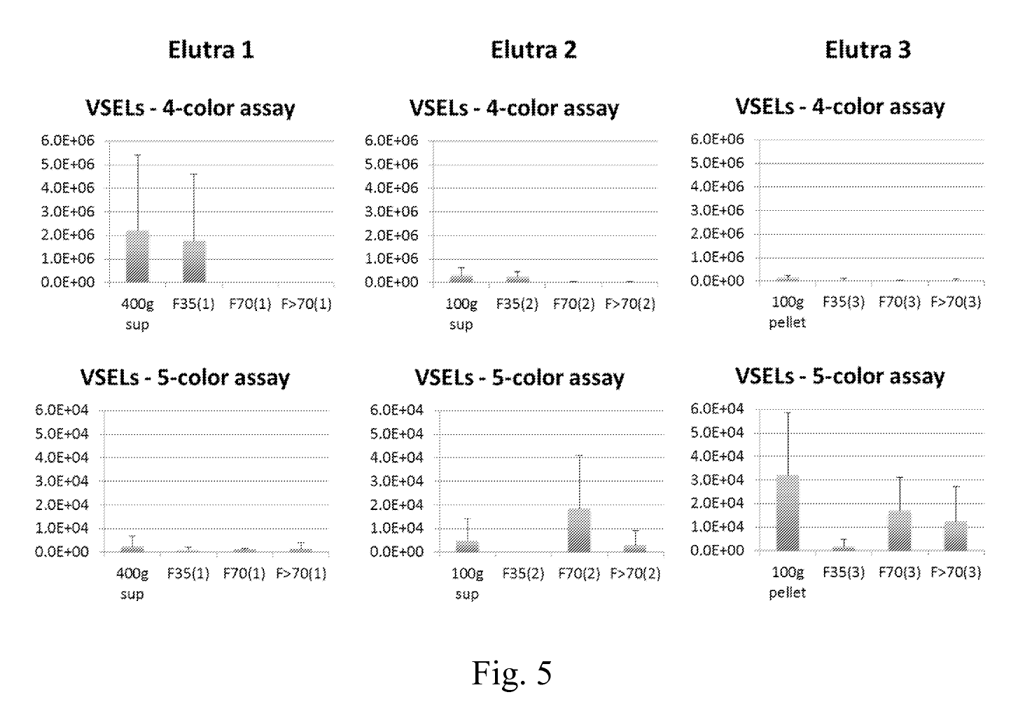

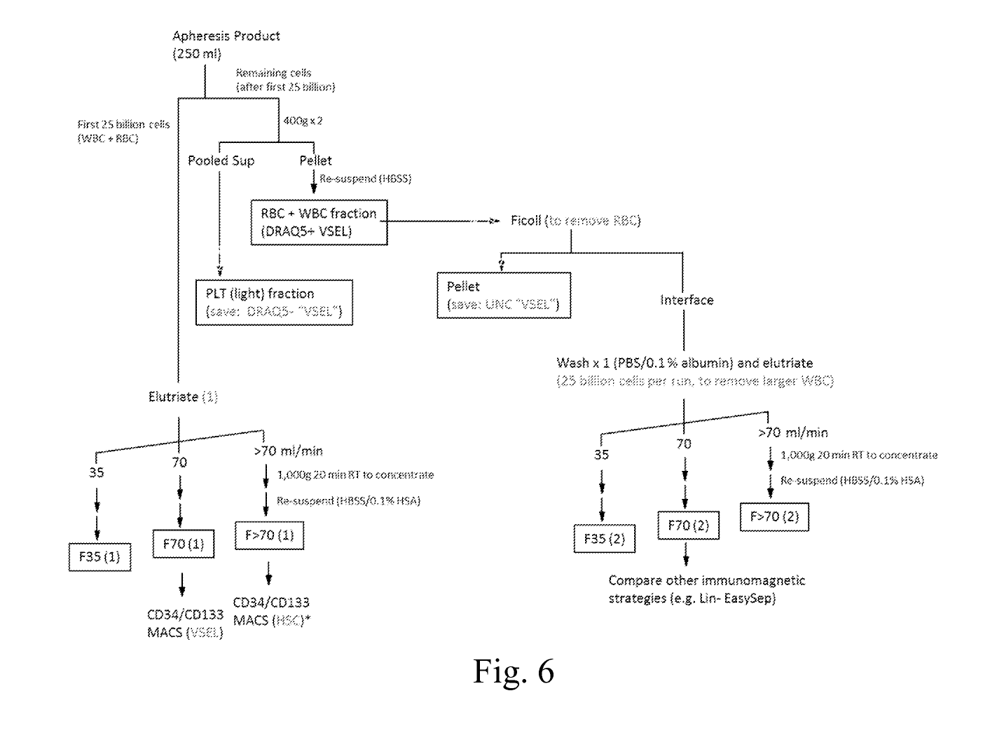

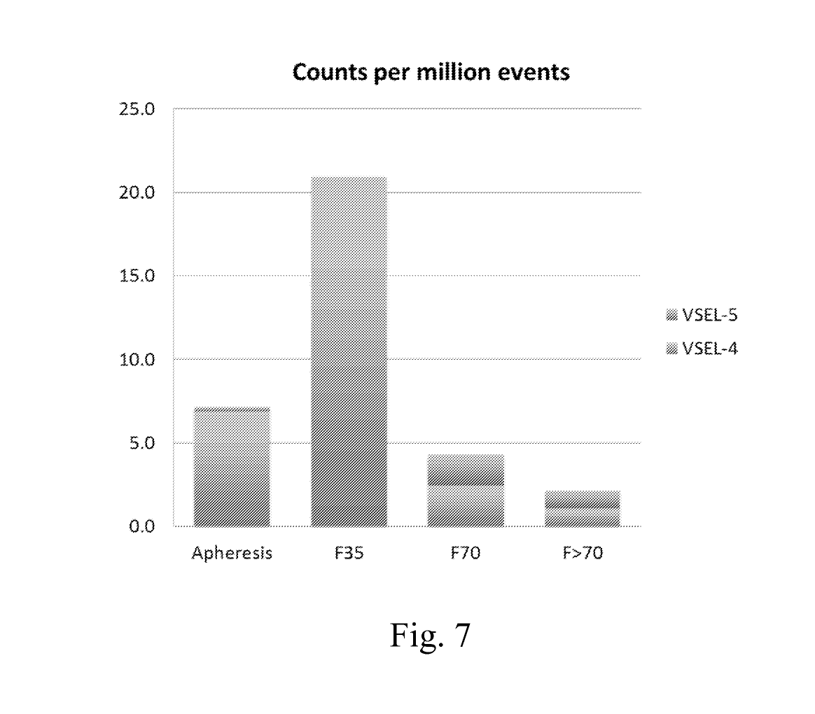

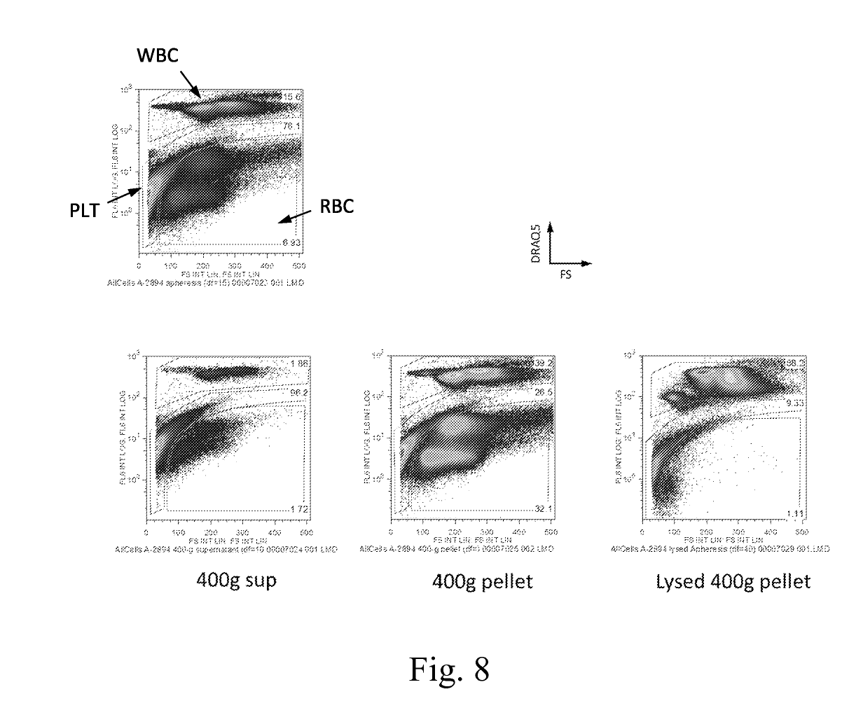

Selective DRAQ5 staining of denser VSEL-like objects was confirmed by centrifugation. FIG. 4 shows that whereas most VSEL events (VSELs+VSEL-like particles) are in a 400 g supernatant, most DRAQ5⁺ VSELs are found in a 100×g pellet. By quantity, DRAQ5⁻ VSELs are about 50× more prevalent than DRAQ5⁺ VSELs. This result was confirmed by elutriation of the pellets and supernatants (FIG. 5). The majority of VSEL-like particles detected by 4-color assay are light and small. VSELs identified by 5-color assay are larger (elutriated in later fractions) and heavier.

### Example 3

### VSEL Enrichment by Immunomagnetic Selection

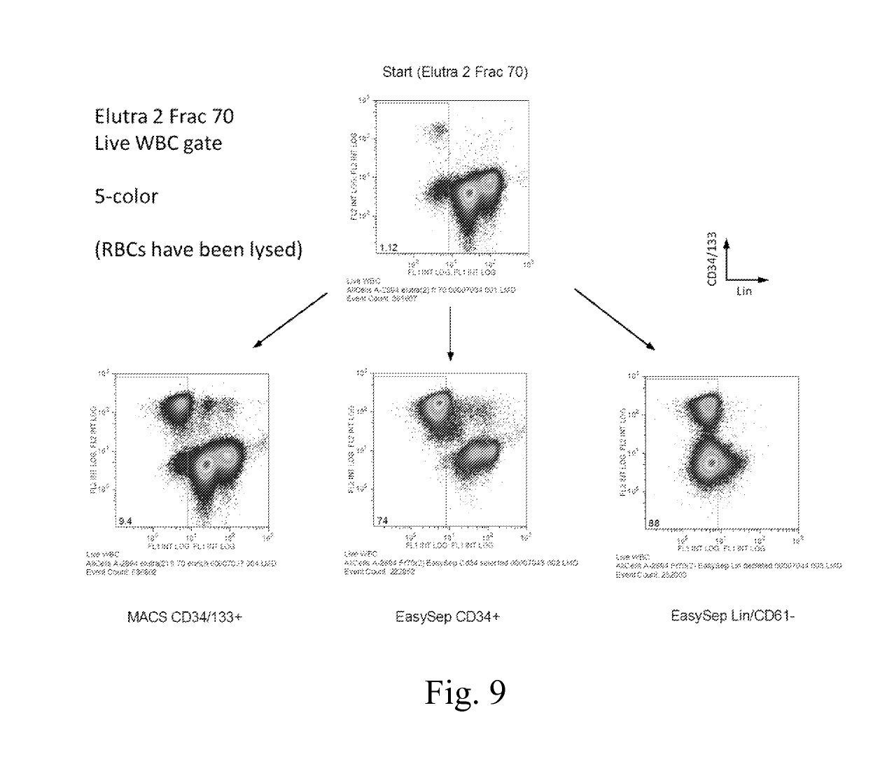

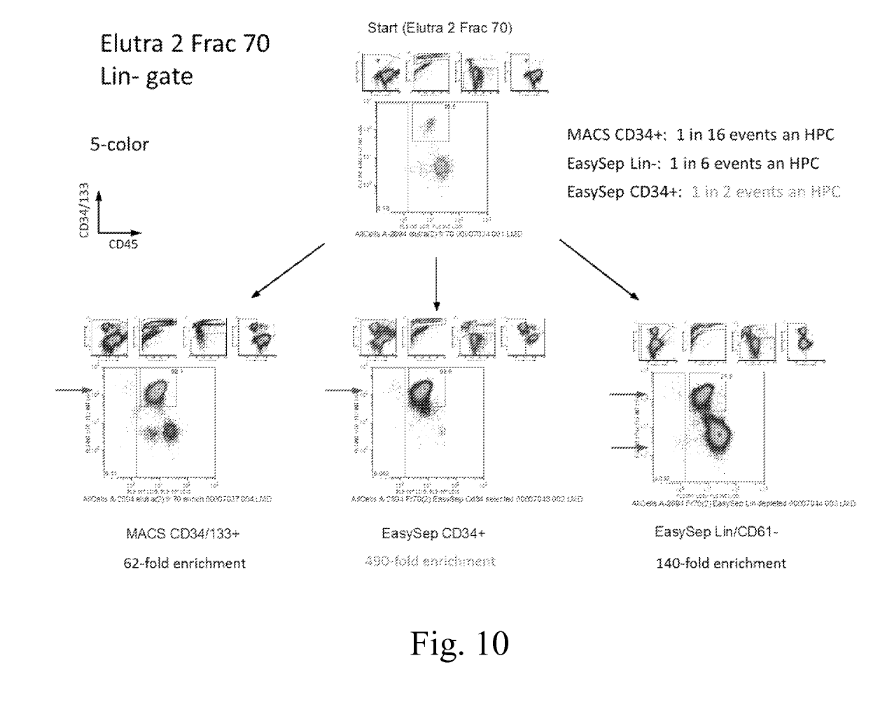

Immunomagnetic selection methods were compared. FIG. 9 shows immunomagnetic enrichment of in the Elutra 2 fraction 70 (elutriation of cells from a 100×g supernatant). RBCs have been lysed. CD34⁺ cells (top center of each plot) are enriched by three different methods (MACS CD34⁺/CD133⁺, EasySep CD34⁺, and EasySep Lin⁻CD61⁻. EasySep CD34⁺ yielded the best enrichment. FIG. 10 shows the lin⁻ gate for the samples depicted in FIG. 9.

### Example 4

### Enrichment of DRAQ5⁻ VSEL-Like Particles from Small/Low-Density Cell Fractions by Immunomagnetic Selection

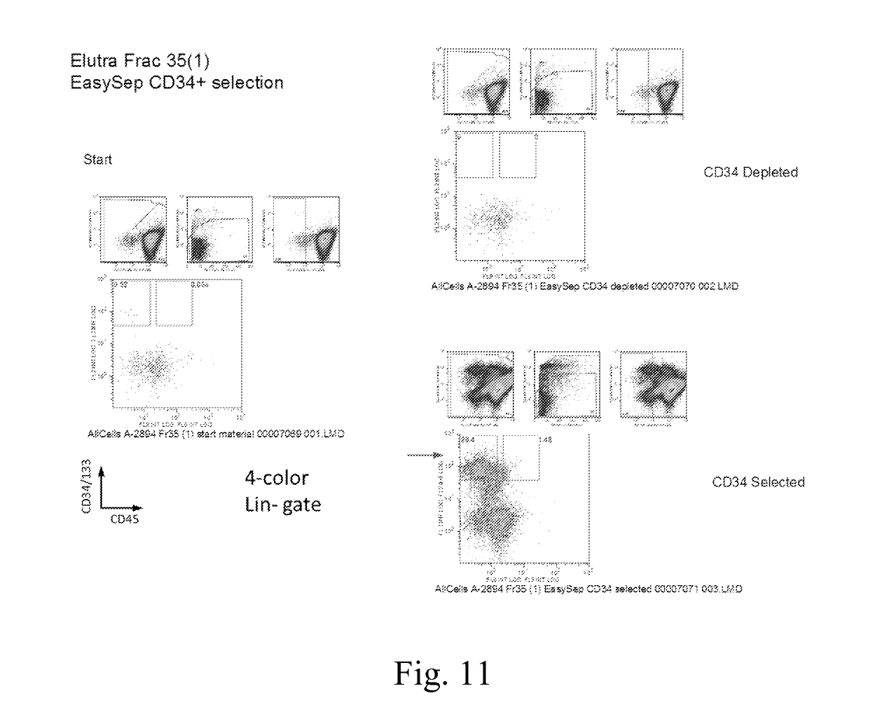

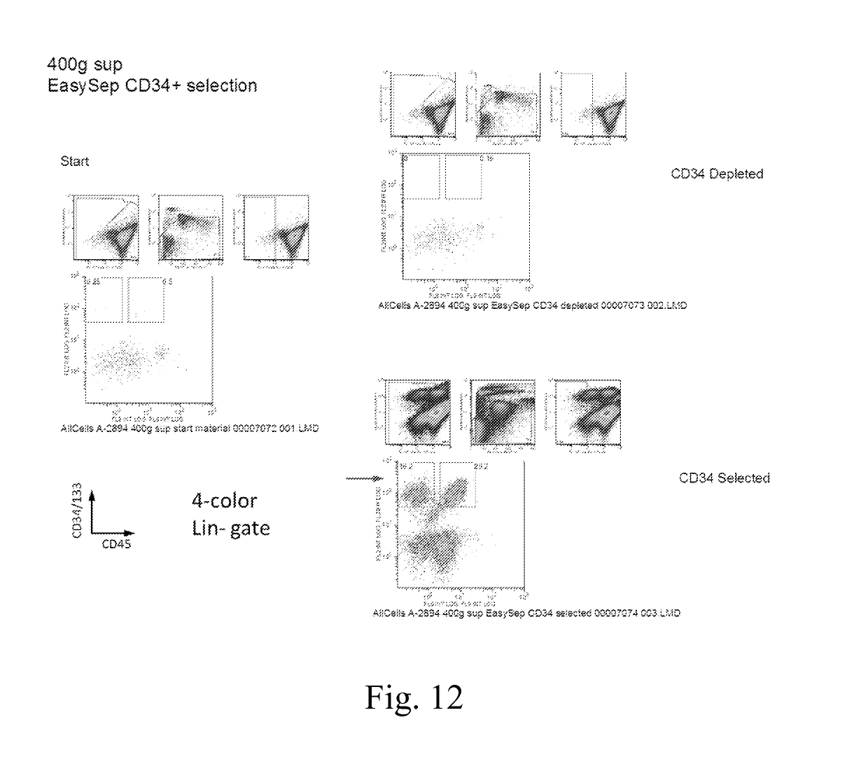

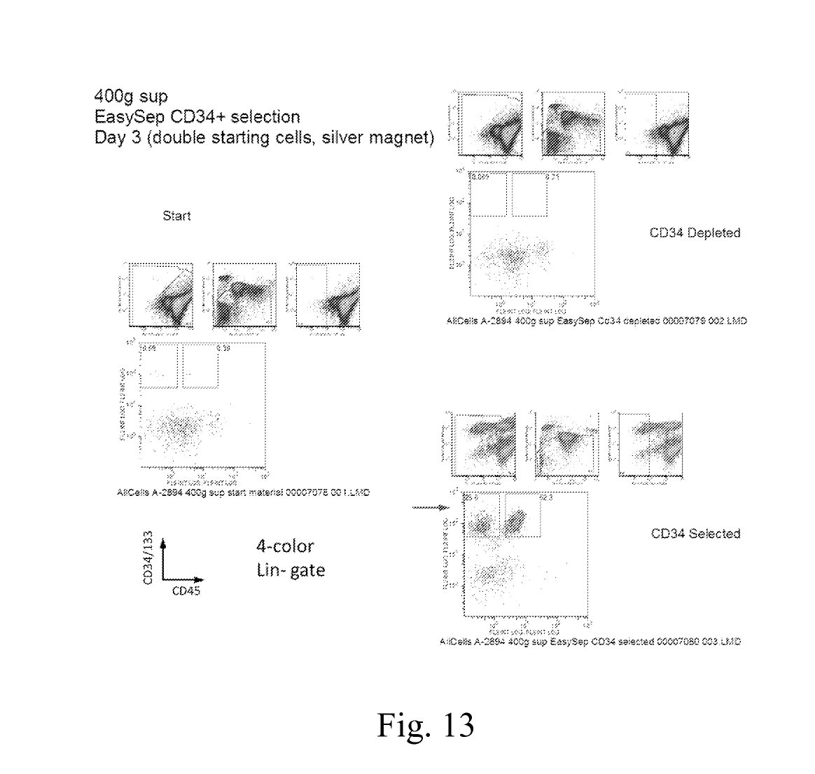

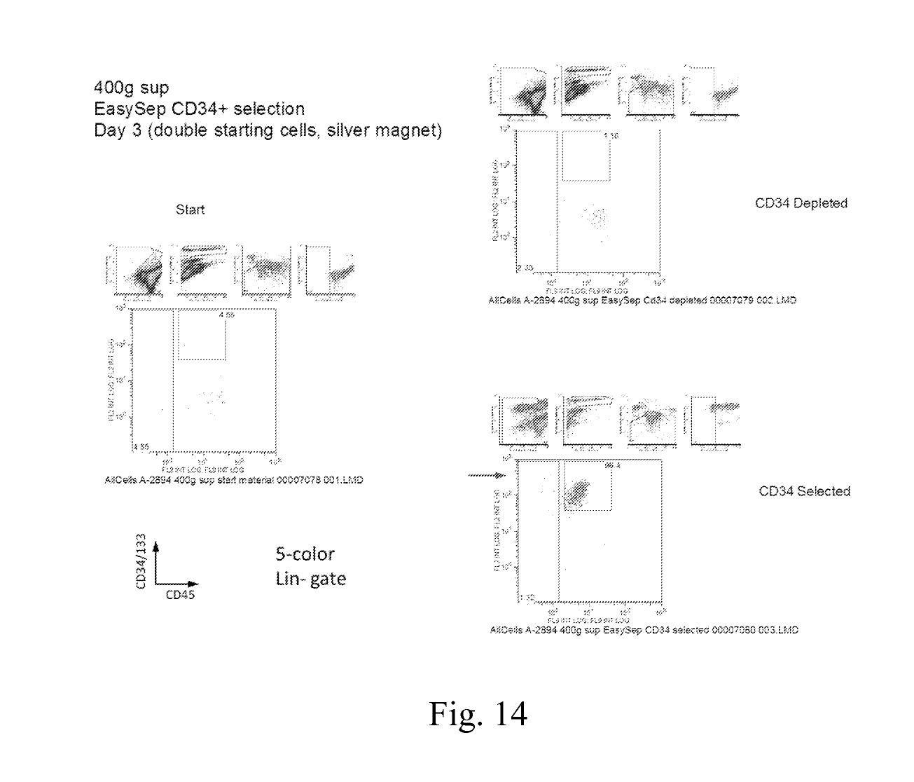

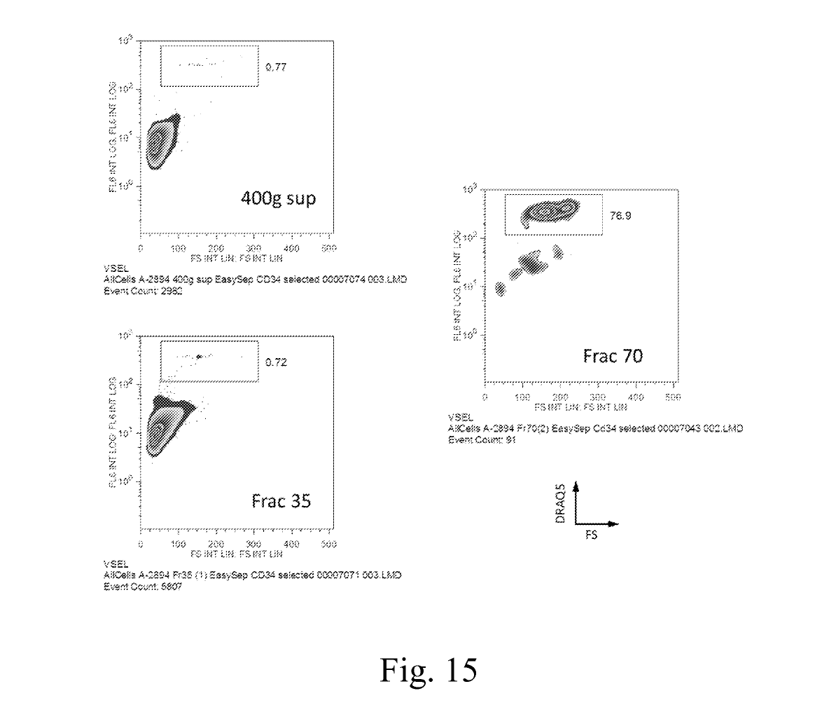

This experiment shows that most small and light VSEL-like particles eluting in fraction 35 are DRAQ5⁻. Material from the 400×g supernatant or separated by elutration (Elutra fraction 35) was analyzed. FIG. 11 shows VSEL-like particles in the Elutra 35 fraction express CD34. FIG. 12 shows VSEL-like particles in the 400×g supernatant express CD34. FIG. 13 shows a second experiment yielding the same results. There was an 800-fold enrichment of VSELs away from the major platelet contaminant in this fraction. As shown in FIG. 14, most of the VSEL events purified from 400×g supernatant are DRAQ5⁻. DRAQ5 labeling was compared among 40×g supernatant, Elutra fraction 35, and Elutra fraction 70. FIG. 15 shows that most DRAQ5 labeled events are particles (cells) that are relatively large and dense.

### Example 5

### VSEL Extracellular Vesicles Stimulate Cell Proliferation and Promote Angiogenesis

Serum-starved HUCECs are incubated with 10 μM BrdU and varying amounts of VSEL extracellular vesicles for 24 hours. The cells are fixed, washed, and incubated with fluorescently labeled anti-BrdU antibodies. HUVEC cell proliferation is found to increase in response to VSEL extracellular vesicle addition in a dose-dependent matter.

HUVECs are cultured on plates coated with Matrigel™ supplemented with VSEL extracellular vesicles and with suitable positive and negative controls (e.g., proangiogenic factors such as SDF-1, VEGF, MSCs, media only), and tube formation is evaluated. Tube formation is stimulated by VSEL extracellular vesicle supplementation.

We claim:

1. A composition comprising enriched extracellular vesicles, wherein the extracellular vesicles comprise CD45⁻/lin⁻/CD34⁺, or CD45⁻/lin⁻/CD133⁺, or CD45⁻/lin⁻/CD34⁺/CD133⁺.

2. The composition of claim 1, wherein the extracellular vesicles are substantially unstained by a vital nuclear dye.

3. The composition of claim 1, wherein the vital nuclear dye is DRAQ5, thiazole orange or LDS-751.

4. The composition of claim 1, wherein the vital nuclear dye is DRAQ5.

5. The composition of claim 1, wherein the composition is substantially free of nucleated cells.

6. The composition of claim 1, wherein the extracellular vesicles comprise CXCR4⁺.

7. The composition of claim 1, wherein the extracellular vesicles comprise at least one of SSEA-4, Oct-4, Rev-1, and Nanog.

8. The composition of claim 1, wherein the extracellular vesicles comprise a lineage-related marker.

9. The composition of claim 1, wherein the extracellular vesicles have a diameter of 40-100 nm, 50-150 nm, 100-300 nm, 200-500 nm, 400-1000 nm, 500-1500 nm, 1-2 μm, 2-3 μm, 3-4 μm, or 4-5 μm

10. A pharmaceutical composition comprising the enriched extracellular vesicles of claim 1.

11. A method for treating damage or injury to a tissue in a subject, comprising administering to the subject an effective amount of a pharmaceutical composition of claim 10.

12. A method of making a composition enriched for extracellular vesicles which express VSEL markers, which comprises:
a) providing a population comprising CD45⁻ extracellular vesicles,
b) identifying a subpopulation of CD45-extracellular vesicles that expresses a marker of VSEL stem cells, and
c) purifying the subpopulation.

13. The method of claim 12, wherein the subpopulation comprises CD45⁻/lin⁻/CD34⁺, or CD45⁻/lin⁻/CD133⁺, or CD45⁻/lin⁻/CD34⁺/CD133⁺.

14. The method of claim 12, wherein the subpopulation is substantially unstained by a vital nuclear dye.

15. The method of claim 12, wherein the subpopulation is substantially unstained by DRAQ5⁻.

16. The method of claim 12, wherein the subpopulation comprises CXCR4⁺.

17. The method of claim 12, wherein the subpopulation comprises at least one of SSEA-4, Oct-4, Rev-1, and Nanog.

18. The method of claim 12, wherein the subpopulation comprises a lineage-related marker.

19. A method of purifying a very small embryonic-like (VSEL) stem cell, which comprises:
a) providing a population comprising CD45⁻ VSEL stem cells,
b) identifying a subpopulation of CD45⁻ stem cells that expresses a marker of VSEL stem cells and stains with a vital nuclear dye, and
c) purifying the subpopulation.

20. The method of claim 19, wherein the vital nuclear dye is DRAQ5, thiazole orange or LDS-751.

21. The method of claim 19, wherein the vital nuclear dye is DRAQ5.

22. The method of claim 19, wherein the VSEL stem cells are substantially unstained by 7-amino-actinomycin D (7-AAD).

23. The method of claim 19, which further comprises identifying and purifying the subpopulation which comprises CD45⁻/lin⁻/CD34⁺, or CD45⁻/lin⁻/CD133⁺, or CD45⁻/lin⁻/CD34⁺/CD133⁺.

24. The method of claim 19, which further comprises identifying and purifying the subpopulation which comprises CXCR4⁺.

25. The method of claim 19, which further comprises identifying and purifying the subpopulation which comprises at least one of SSEA-4, Oct-4, Rev-1, and Nanog.
